(19) United States
(12) Patent Application Publication
(10) Pub. No.: US 2012/0220664 A1
(43) Pub. Date: Aug. 30, 2012

(54) USE OF METFORMIN IN CANCER TREATMENT AND PREVENTION

(75) Inventors: Kevin Struhl, Weston, MA (US); Heather Hirsch, Boston, MA (US); Dimitrios Iliopoulos, Brighton, MA (US)

(73) Assignee: PRESIDENT AND FELLOWS OF HARVARD COLLEGE, Cambridge, MA (US)

(21) Appl. No.: 13/391,890

(22) PCT Filed: Aug. 25, 2010
(86) PCT No.: PCT/US10/46616

§ 371 Date: May 7, 2012

Related U.S. Application Data
(60) Provisional application No. 61/236,778, filed on Aug. 25, 2009.

(51) Int. Cl. A61K 31/155; A61P 35/00

(52) U.S. Cl. 514/635

(57) ABSTRACT

Disclosed herein is a method for treating a tumor in a subject in need thereof comprising administering an enhancing amount of metformin and a reduced amount of one or more chemotherapeutic agents. One example of an enhancing amount of metformin is about 250 mg/day. Also disclosed is a Untreated method for preventing cancer or delaying the recurrence of cancer in a subject comprising administering an effective amount of metformin to the subject. In one example of such a method, the amount of metformin is about 75 mg/day. Also disclosed is a composition comprising an enhancing amount of metformin, and a reduced amount of one or more chemotherapeutic agents and a pharmaceutically acceptable carrier. Kits comprising metformin and one or more chemotherapeutic agents are also disclosed.

### RELATED APPLICATIONS

This application claims the benefit of priority under 35 U.S.C. §119(e) to U.S. Provisional patent application Ser. No. 61/236,778, filed Aug. 25, 2009, the contents of which are herein incorporated by reference in their entirety.

### GOVERNMENTAL SUPPORT

This invention was made with Government support under CA 57436 and CA 107486 awarded by the National Institutes of Health. The Government has certain rights in the invention.

### FIELD OF THE INVENTION

The present invention relates to the field of tumor therapy.

### BACKGROUND OF THE INVENTION

Chemotherapeutic treatments for cancer can effectively reduce tumor mass, but the disease often relapses. To explain this phenomenon, the cancer stem cell hypothesis suggests that tumors contain a small number of tumor-forming, self-renewing, cancer stem cells within a population of non-tumor-forming cancer cells (1, 2). Unlike most cells within the tumor, cancer stem cells are resistant to well-defined chemotherapy, and after treatment, they can regenerate all the cell types in the tumor through their stem cell-like behavior. For this reason, drugs that selectively target cancer stem cells offer great promise for cancer treatment, although none are known at present.

### SUMMARY OF THE INVENTION

One aspect of the present invention relates to a method for treating a cancer/tumor in a subject in need thereof comprising administering an enhancing amount of metformin and a reduced amount of one or more chemotherapeutic agents. In one embodiment, the enhancing amount of metformin is 250 mg/day.

Another aspect of the present invention relates to a composition comprising an enhancing amount of metformin, and a reduced amount of one or more chemotherapeutic agents and a pharmaceutically acceptable carrier. In one embodiment, the enhancing amount of metformin is the enhancing amount of metformin is about 25 mg, 75 mg, or 250 mg.

Another aspect of the present invention relates to a method for preventing cancer or delaying the recurrence of cancer in a subject comprising administering an effective amount of metformin to the subject. In one embodiment, the amount of metformin is the amount of metformin is about 75 mg/day.

Another aspect of the present invention relates to a kit comprising a vial or metformin, a vial of one or more chemotherapeutic agents, instructions for the use of the metformin and the chemotherapeutic agent(s) together.

### BRIEF DESCRIPTION OF THE DRAWINGS

FIG. 1A-FIG. 1B contain graphical representations of data from experiments, the results of which indicate that metformin prevents transformation of MCF10A-ER-Src cells. FIG. 1A: Number of cells grown in the presence or absence of 1 μM 4-hydroxy tamoxifen (TAM) with the indicated concentrations of metformin for 24 hours. FIG. 1B: Relative number of foci, colonies in soft agar, and mammospheres in untreated or TAM-treated cells in the presence of the indicated concentration of metformin.

FIG. 2 is a bar graph of data from experiments, the results of which indicate that metformin inhibits growth of mammospheres. 6-day old mammospheres from the indicated cell lines were or were not treated with 0.1 mM metformin for 48 hr, and the number of mammospheres counted.

FIG. 3A-FIG. 3C contain graphical representations of data from experiments, the results of which indicate that metformin selectively kills cancer stem cells and functions synergistically with doxorubicin. FIG. 3A: Number of cancer stem cells (CD44high/CD24low; black) and cancer cells (CD44low/CD24high; grey) in the transformed (36 h TAM treatment) MCF-10A population that was treated with doxorubicin, 0.1 mM metformin, or both (n=3). FIG. 3B: Cancer stem cells (SC) and non-stem cancer cells (NSC) obtained by sorting were treated with 0.1 mM metformin for 0, 24, and 48 hours. FIG. 3C: Tumor volume in nude mice at the indicated number of days after injection of MCF10A-ER-Src cancer stem cells that were or were not treated with 0.1 mM metformin for 1 hr prior to injection.

FIG. 4A-FIG. 4B contains graphical representations of data from experiments, the results of which indicate that metformin and doxorubicin act in combination to reduce tumor mass and prolong remission in nude mice. FIG. 4A: Tumor volume (mean values and 95% confidence intervals) of mice injected with transformed MCF10A-ER-Src cells (time 0 indicates the time of injection) that were untreated, or treated by intraperitoneal injections every 5 days (3 cycles; arrows indicate the day or injections) with 4 mg/kg doxorubicin (Dox), 100 μg/ml metformin (Met), or both. FIG. 4B: Number of cancer stem cells (CD44high/CD24low) in cells obtained from tumors treated with Dox or the combination of Dox+Met after 3 cycles of treatment (day 25).

### DETAILED DESCRIPTION OF THE INVENTION

The cancer stem cell hypothesis suggests that, unlike most cancer cells within a tumor, cancer stem cells resist chemotherapeutic drugs and can regenerate the various cell types in the tumor, thereby causing relapse of the disease. Thus, drugs that selectively target cancer stem cells offer great promise for cancer treatment, particularly in combination with chemotherapy. Here, we show that low doses of metformin, a standard drug for diabetes, inhibits cellular transformation and selectively kills cancer stem cells in four genetically different types of breast cancer. The combination of metformin and a well-defined chemotherapeutic agent, doxorubicin, kills both cancer stem cells and non-stem cancer cells in culture. Furthermore, this combinatorial therapy reduces tumor mass and prevents relapse much more effectively than either drug alone in a xenograft mouse model. Mice remain tumor-free for at least two months after combinatorial therapy with metformin and doxorubicin is ended. These results provide further evidence supporting the cancer stem cell hypothesis, and they provide a rationale and experimental basis for using the combination of metformin and chemotherapeutic drugs to improve treatment of patients with breast and other cancers.

Aspects of the present invention are based on the findings that metformin enhances the anti-tumor effects of chemotherapeutic agents used in therapeutic treatments (e.g., cancer therapy). As such, the amount of the chemotherapeutic agent required to produce the therapeutic anti-tumor effects is reduced. Reduction in the amount of the chemotherapeutic agent results in decreased side effects to the recipient from the chemotherapeutic agent. Accordingly, one aspect of the present invention is directed to a method of increasing anti-tumor effect of a chemotherapeutic agent, the method comprising administering to a patient in need thereof an enhancing amount of metformin and a reduced amount of a chemotherapeutic agent.

### Definitions

As used herein, the phrase “cytotoxic agent” means an agent used to treat abnormal and uncontrolled progressive cellular growth. Preferred cytotoxic agents include, for example, cyclophosphamide, ifosfamide, cytarabine, 6-mercaptopurine, 6-thioguanine, vincristine, doxorubicin, and daunorubicin, chlorambucil, carmustine, vinblastine, methotrexate, and paclitaxel.

As the term is used herein, an “enhancing amount” of metformin is an amount sufficient to produce a statistically significant, reproducible enhancement of the anti-tumor effects of a chemotherapeutic agent (e.g., cytotoxic agent) or therapy (e.g., radiation therapy). Enhancement of an anti-tumor effect can be determined through a variety of means known in the art. For example, enhancement of an anti-tumor effect can be determined through a statistically significant decrease in the administered amount of the agent or therapy required to produce the anti-tumor effects. This is determined for example, by comparison to an appropriate control group receiving a standard amount of the therapy in the absence of metformin.

As the term is used herein, a “chemotherapeutic agent” refers to an chemical or drug used in the treatment of a tumor. Such agents are often cytotoxic agents.

As the term is used herein, “radiation therapy” refers to the use of ionizing radiation to kill cancer cells and shrink tumors.

As the term is used herein, a “reduced amount” of a chemotherapeutic agent (e.g., a cytotoxic agent) or therapy is an amount which is less than the standard amount administered to a subject suffering from a tumor for treatment of the tumor, to produce the same or better therapeutic results. One benefit of using a reduced amount of a chemotherapeutic agent or tumor therapy is a reduction in the side effects experienced by the recipient, with the same or increased therapeutic results. Reduction in the amount can be a reduction in the amount given at one or more individual administration (dosage), a reduction in the frequency of administration, or a combination thereof. Guidance for standard dosages and administration schedules regimens are provided in the art to the skilled practitioner (e.g, in the Physicians' Desk Reference, 56.sup.th Ed. (2002) Publisher Edward R. Barnhart, N.J. (“PDR”)). The reduced amount is markedly lower than a standard dose commonly used in therapeutic administration (e.g., reduced to about 90%, 80%, or 70% of the standard dosage). In some instances, therapeutic benefit will be obtained from administration of a dosage amount that is a reduction of the standard dosage to less than 75% (e.g., administration is within about 75% to 25% of the standard dosage). Therapeutic benefit is expected to be obtained from administration of a dosage amount that is a reduction of the standard dosage to about 60%, 50%, or 40% of the standard dosage. In some instances, therapeutic benefit will be obtained from administration of a dosage amount that is a reduction of the standard dosage to less than 40% (e.g., administration is within about 40% to 10% of the standard dosage). In one embodiment, the dosage is about 30% of the standard dosage. In one embodiment, the dosage is about 20% of the standard dosage. In one embodiment, the dosage is about 10% of the standard dosage.

As the term is used herein, an “anti-tumor effect” or “anti-cancer effect” refers to reduction in tumor growth, growth rate, size, spread, metastasis, as well as prevention of occurrence and/or recurrence of a tumor in an individual.

By the term “treat” as in “treat a subject,” is meant to give medical aid to such subject especially, for the purposes of preventing the development of, or preventing the worsening of an undesired physiological or medical condition, or for the purposes of ameliorating such condition in such subject, either human or animal. Unless otherwise stated, the term “treat” is not limited to any particular length of time or to any particular level of dose.

The terms “composition” or “pharmaceutical composition” are used interchangeably herein and refers to compositions or formulations that usually comprise an excipient, such as a pharmaceutically acceptable carrier that is conventional in the art and that is suitable for administration to a subject. Such compositions can be specifically formulated for administration via one or more of a number of routes, including but not limited to, oral, ocular and nasal administration and the like.

The “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and is compatible with administration to a subject, for example a human.

As used herein, a “subject” refers to an animal such as a mammal, avian, reptile, amphibian or fish. The term “mammal” is intended to encompass a singular “mammal” and plural “mammals,” and includes, but is not limited: to humans, primates such as apes, monkeys, orangutans, and chimpanzees; canids such as dogs and wolves; fields such as cats, lions, and tigers; equids such as horses, cows, donkeys, and zebras, food animals such as cows, pigs, and sheep; ungulates such as deer and giraffes; rodents such as mice, rabbits, rats, hamsters and guinea pigs.

The term “individual”, “subject”, and “patient” are used interchangeably herein, and refer to an animal, for example a mammal, such as a human.

The term “metformin” as employed herein refers to metformin or a pharmaceutically acceptable salt thereof such as the hydrochloride salt, the metformin (2:1) fumarate salt, and the metformin (2:1) succinate salt as disclosed in U.S. application Ser. No. 09/262,526 filed Mar. 4, 1999, the hydrobromide salt, the p-chlorophenoxy acetate or the embonate, and other known metformin salts of mono and dibasic carboxylic acids including those disclosed in U.S. Pat. No. 3,174,901, all of which salts are collectively referred to as metformin. The metformin employed herein may be the metformin hydrochloride salt, namely, that marketed as Glucophage.®. (trademark of Bristol-Myers Squibb Company).

The term “derivative” as used herein, refers to compounds with similar chemical structure and similar function.

In this specification and the appended claims, the singular forms “a,” “an,” and “the” include plural references unless the context clearly dictates otherwise. Thus, for example, reference to a composition for delivering “a drug” includes reference to two or more drugs. In describing and claiming the present invention, the following terminology will be used in accordance with the definitions set out below.

Another aspect of the invention relates to a method of reducing the side effects of a chemotherapeutic agent during treatment of a subject for a tumor, the method comprising administering to the subject an enhancing amount of metformin and a reduced amount of the chemotherapeutic, wherein the amount of chemotherapeutic agent administered causes less side effects compared to a conventional amount of the chemotherapeutic agent. The invention is further directed to a tumor inhibiting pharmaceutical composition comprising an enhancing amount of metformin and a reduced amount of one or more chemotherapeutic agents, wherein the tumor inhibiting amount of the chemotherapeutic agent(s) is an amount that results in decreased side effects.

In one embodiment of the methods described herein, the chemotherapeutic agent is not inhibitory to cancer stem cells. In another embodiment of the methods described herein, the chemotherapeutic agent is inhibitory to cancer stem cells. Combination therapies with cancer stem cell inhibitory and non-inhibitory chemotherapeutics is also envisioned.

In one embodiment, the enhancing amount of metformin is administered with a cocktail of standard chemotherapeutic agents (e.g., in reduced amounts), to a patient following surgery for removal of a tumor (e.g., cancer).

The effect of co-administration of metformin, as discussed herein, is also expected to enhance other forms of anti-tumor therapies, (e.g., hormonal therapies such as interferon therapy, or radiation therapy). As such, the therapeutic methods described herein for the use of chemotherapeutic agents can alternatively be performed using enhancing amounts of metformin and using reduced amounts of administration and/or frequencies of these other forms of tumor therapies and their corresponding therapeutic agents as well. The use of enhancing amounts of metformin with combinations of one or more therapies and/or in combination with with one or more other therapeutics described herein is also envisioned.

The effect of co-administration of metformin, as discussed herein, is further expected to enhance the effects of other agents that kill tumor/cancer cells. This includes other agents (e.g., drugs) that are not traditionally part of chemotherapy, such as drugs that affect transformation of the cells (e.g., Exendin4, aspirin, meloxicam, indomethacin, celecoxib, piroxican, nimesulfide, sulindac, tocilizumab, simvastatin, cerulenin, mevastatin). Such agents can be tested in assays (e.g., the cell assays described herein) for synergy/enhancement by metformin co-administration. As such, the therapeutic methods described herein for the use of chemotherapeutic agents can alternatively be performed using enhancing amounts of metformin and using reduced amounts of administration and/or frequencies of these other tumor/cancer killing agents. Such agents that would kill tumor/cancer cells, include, without limitation, antibodies (e.g., anti-HER2), tamoxifen and other compounds that inhibit transformation. The use of enhancing amounts of metformin with combinations of one or more therapies and/or agents, and/or in combination with with one or more other therapeutics, and/or agents described herein is also envisioned.

The ability of metformin to enhance a given chemotherapetuic agent or treatment thereby allowing a reduced amount to be given to a subject, is within the ability of the skilled practitioner. For example, enhancement of a chemotherapeutic agent or tumor treatment is evidenced by increased efficacy of the agent or treatment when in combination with metformin administration, as compared to one or more appropriate controls lacking metformin administration. Efficacy of treatment can be judged by an ordinarily skilled practitioner. Efficacy can be assessed in animal models of cancer and tumor, for example treatment of a rodent with a cancer, and any treatment or administration of the compositions or formulations that leads to a decrease of at least one symptom of the tumor, for example a reduction in the size of the tumor or a slowing or cessation of the rate of growth of the tumor indicates effective treatment.

Efficacy for any given formulation can also be judged using an experimental animal model of cancer, e.g., wild-type mice or rats, or preferably, transplantation of tumor cells akin to that described in the Examples herein below. When using an experimental animal model, efficacy of treatment is evidenced when a reduction in a symptom of the tumor, for example a reduction in the size of the tumor or a slowing or cessation of the rate of growth of the tumor occurs earlier in treated, versus untreated animals. By “earlier” is meant that a decrease, for example in the size of the tumor occurs at least 5% earlier, but preferably more, e.g., one day earlier, two days earlier, 3 days earlier, or more.

Experiments detailed in the Examples section below indicate that metformin selectively kills cancer stem cells, and that this killing occurs when the cancer stem cells are exposed to relatively low concentrations of the metformin. As such, another aspect of the invention relates to a method for preventing or delaying the development of a tumor/cancer in a subject comprising administering metformin to the subject. Such a subject may be, for example, predisposed for tumor development (e.g., genetically or due to exposure to carcinogenic agents). Without limitation, examples of such genetic predispositions include predisposing mutations in the brca1, brcaII, rb,or p53 gene. In one embodiment, the subject has previously received chemo or radiation therapy and is at high risk for developing a secondary cancer. One such example is a subject who was treated for childhood leukemia or lymphoma. In one embodiment, the metformin is administered by the methods described herein, to contact a precancerous lesion (e.g., a skin lesion) to thereby prevent the lesion from developing into cancer. In another embodiment, the metformin is administered following removal of such a lesion (e.g., to contact the site of removal).

Another aspect of the present invention relates to the treatment of bone marrow or peripheral blood bone marrow stem cells samples with metformin prior to autologous transplants in the treatment of blood cancer, to thereby reduce cancer stem cells. Such treatment will decrease the likelihood of reseeding stem cells. In one embodiment, the metfomin can be administered to the subject receiving the transplant after the transplant has taken place (e.g, for days, weeks, months, or a year following the transplant.)

Another aspect of the present invention relates to the administration of low doses of metformin for long-term cancer prevention in a subject. In one embodiment, such administration is in the form of a dietary supplement or a regular food supplemented with the metformin (e.g., formulated animal food such as dog food, cat food, or food routinely given to farm animals). Such formulations of food and dietary supplements are also encompassed by the present invention.

Another aspect of the present invention relates to an assay for testing derivatives of metformin for the ability to enhance chemotherapeutic agents, tumor killing agents, and other therapies, in killing cancer cells. The cell assays in described in the Examples section below can be adapted for such assays by the skilled artisan.

### Dosage and Administration

In therapeutic applications, the standard dosages and administration schedule of the chemotherapeutic agent or therapy used can vary depending on a number of variables, such as combinations of cytotoxic agents or therapies being administered, the tumor type, the age, weight, and clinical condition of the recipient patient, the route of administration, and the experience and judgment of the clinician or practitioner administering the therapy. However, the present invention allows for such a dosage and/or administration schedule to be reduced significantly, thereby resulting in decreased side effects from the treatment.

In one embodiment, the amount of metformin adminstered may be a standard dose commonly used in therapeutic administration for treatment of type 2 diabetes (from about 1500 mg/day to about 2550 mg/day). In another embodiment, the therapeutic amount of metformin (e.g., used in enhancing tumor treatment with a chemotherapeutic, or used in prevention of tumor development) is markedly lower than a standard dose commonly used in therapeutic administration for treatment of type 2 diabetes (e.g., reduced to about 90%, or about 1350 mg/day, 80%, or about 1200 mg/day, or 70%, or about 1050 mg/day, of the standard dosage). In some instances, therapeutic benefit will be obtained from administration of a dosage amount that is a reduction of the standard dosage to less than 75% (e.g., administration is within about 75%, or about 1125 mg/day, to 25%, or about 375 mg/day, of the standard dosage). Therapeutic benefit is expected to be obtained from administration of a dosage amount that is a reduction of the standard dosage to about 60%, or about 900 mg/day, 50%, or about 750 mg/day, or 40%, or about 600 mg/day, of the standard dosage. In some instances, therapeutic benefit will be obtained from administration of a dosage amount that is a reduction of the standard dosage to less than 40% (e.g., administration is within about 40% to 10% of the standard dosage, or from about 600 mg/day to about 150 mg/day,). In one embodiment, the dosage is about 30%, or about 450 mg/day, of the standard dosage. In one embodiment, the dosage about is 20%, or about 300 mg/day, of the standard dosage. In one embodiment, the dosage is about 10%, or about 150 mg/day, of the standard dosage.

Administration is performed such that the administered agents (e.g., metformin and chemotherapeutic agent) contact the tumor or the tumor site (e.g., after removal of the tumor). Suitable routes of administration are known in the art. The agents described herein may be administered in any manner found appropriate by a clinician, such as those described in the Physicians' Desk Reference, 56.sup.th Ed. (2002) Publisher Edward R. Barnhart, N.J. (“PDR”). For example, parenterally, enterally, topically. The combined agents, or each agent individually can be administered by any means known in the art. Such modes include oral, rectal, nasal, topical (including buccal and sublingual), or parenteral (including subcutaneous, intramuscular, intravenous, and intradermal) administration. The metformin and enhanced agent can be adminsitered systemically, or can be administered locally to the, or near the tumor site (e.g, by injecton into the tumor or an organ or part of the body containing the tumor). In one embodiment, the metformin and enhanced therapeutic agents (e.g, chemotherapeutic agents) are adminstered into the central nervous system.

Administration can be pre-operative or post-operative, or both. In one embodiment, the metformin is adminstered three times a day (e.g., 25 mg/dose) for one month before surgery and removal of the tumor.

Administration of metformin (when applicable with a chemotherapeutic agent) in the methods described herein can be for extended period of time (e.g, 6-12 months, or 1, 2, 3 years, or indefinitely). In one embodiment, the metformin is administered more often than the chemotherapeutic agent. For example, a subject can be administered the chemotherapeutic agent(s) (e.g., doxorubicin), at a signfiicantly reduced frequency than otherwise prescribed, such as 3 days/month, while being administered metformin (eg., 250 mg/day) on a daily basis.

Generally, the dose and administration scheduled should be sufficient to result in slowing, and preferably regressing, the growth of the tumor(s) and also preferably causing complete regression of the tumor. In some cases, regression can be monitored by a decrease in blood levels of tumor specific markers. An effective amount of a pharmaceutical agent is that which provides an objectively identifiable improvement as noted by the clinician or other qualified observer. Regression of a tumor in a patient is typically measured with reference to the diameter of a tumor. Decrease in the diameter of a tumor indicates regression. Regression is also indicated by failure of tumors to reoccur after treatment has stopped.

The metformin and chemotherapeutic agents in combination, or separately, are delivered at periodic intervals that can range from several times a day to once per month. As noted above, the agents are administered until the desired therapeutic outcome has been obtained. Additionally, in order to avoid side-effects, not all components of the combination may require delivery at each administration.

### Therapeutic Agents

Currently available cytotoxic drugs can be broadly divided by their mechanism of action into four groups: alkylating agents, anti-metabolites, antibiotics, and miscellaneous other activities. The choice of a particular cytotoxic agent to treat an individual with cancer is influenced by many factors, including the type of cancer, the age and general health of the patient, and issues of multidrug resistance.

The composition of the invention can utilize a variety of cytotoxic agents, including but not limited to the following agents (including possible sources): the alkylating agents cyclophosphamide (Bristol-Meyers Squibb), ifosfamide (Bristol-Meyers Squibb), chlorambucil (Glaxo Wellcome), and carmustine (Bristol-Meyers Squibb); the anti-metabolites cytarabine (Pharmacia & Upjohn), 6-mercaptopurine (Glaxo Wellcome), 6-thioguanine (Glaxo Wellcome), and methotrexate (Immunex); the antibiotics doxorubicin (Pharmacia & Upjohn), daunorubicin (NeXstar), and mitoxantrone (Immunex); and miscellaneous agents such as vincristine (Lilly), vinblastine (Lilly), and paclitaxel (Bristol-Meyers Squibb). Preferred cytotoxic agents include cyclophosphamide, ifosfamide, cytarabine, 6-mercaptopurine, 6-thioguanine, doxorubicin, daunorubicin, mitoxantrone, and vincristine. The most preferred cytotoxic agent are cyclophosphamide and ifosfamide.

Chemotherapeutic agents are known in the art and include at least the taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, adrenocortical suppressants, or antagonists. More specifically, the chemotherapeutic agents may be one or more agents chosen from the non-limiting group of steroids, progestins, estrogens, antiestrogens, or androgens. Even more specifically, the chemotherapy agents may be azaribine, bleomycin, bryostatin-1, busulfan, carmustine, chlorambucil, carboplatin, cisplatin, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, paclitaxel, phenyl butyrate, prednisone, procarbazine, semustine streptozocin, tamoxifen, taxanes, taxol, testosterone propionate, thalidomide, thioguanine, thiotepa, uracil mustard, vinblastine, or vincristine. The use of any combinations of chemotherapy agents is also contemplated.

Other suitable therapeutic agents are selected from the group consisting of radioisotope, boron addend, immunomodulator and chemosensitizing agent (See, U.S. Pat. Nos. 4,925,648 and 4,932,412). Suitable chemotherapeutic agents are described in REMINGTON'S PHARMACEUTICAL SCIENCES, 19th Ed. (Mack Publishing Co. 1995), and in Goodman and Gilman's The Pharmacological Basis of Therapeutics (Goodman et al., Eds. Macmillan Publishing Co., New York, 1980 and 2001 editions). Other suitable chemotherapeutic agents, such as experimental drugs, are known to those of skill in the art. It is well known in the art that various methods of radionuclide therapy can be used for the treatment of cancer and other pathological conditions, as described, e.g., in Harbert, “Nuclear Medicine Therapy”, New York, Thieme Medical Publishers, 1087, pp. 1-340. Moreover a suitable therapeutic radioisotope is selected from the group consisting of α-emitters, β-emitters, γ-emitters, Auger electron emitters, neutron capturing agents that emit α-particles and radioisotopes that decay by electron capture. Preferably, the radioisotope is selected from the group consisting of 225Ac, 198Au, 32P, 125I, 131I, 90Y, 186Re, 188Re, 67Cu, 177Lu, 213Bi, 10B, and 211At.

In another embodiment, different isotopes that are effective over different distances as a result of their individual energy emissions are used as first and second therapeutic agents. Such agents can be used to achieve more effective treatment of tumors, and are useful in patients presenting with multiple tumors of differing sizes, as in normal clinical circumstances.

Few of the available isotopes are useful for treating the very smallest tumor deposits and single cells. In these situations, a drug or toxin may be a more useful therapeutic agent. Accordingly, in preferred embodiments of the present invention, isotopes are used in combination with non-isotopic species such as drugs, toxins, and neutron capture agents. Many drugs and toxins are known which have cytotoxic effects on cells, and can be used in connection with the present invention. They are to be found in compendia of drugs and toxins, such as the Merck Index, Goodman and Gilman, and the like, and in the references cited above.

Drugs that interfere with intracellular protein synthesis can also be used in the methods of the present invention; such drugs are known to those skilled in the art and include puromycin, cycloheximide, and ribonuclease.

### Radiation Therapy

A variety of radiation therapies are used in tumor therapy. Applicants envision the use of enhancing amounts of metaformin to allow reduced amounts of any one or a combination of such radiation therapies in tumor treatment.

For some types of tumors, radiation may be given to areas that do not have evidence of tumors. This is done to prevent tumor cells from growing in the area receiving the radiation. This technique is called prophylactic radiation therapy. Radiation therapy also can be given to help reduce symptoms such as pain from cancer that has spread to the bones or other parts of the body. This is called palliative radiation therapy.

Radiation may come from a machine outside the body (external radiation), may be placed inside the body (internal radiation), or may use unsealed radioactive materials that go throughout the body (systemic radiation therapy). The type of radiation to be given depends on the type of cancer, its location, how far into the body the radiation will need to go, the patient's general health and medical history, whether the patient will have other types of cancer treatment, and other factors. Most people who receive radiation therapy for cancer have external radiation. Some patients have both external and internal or systemic radiation therapy, either one after the other or at the same time. External radiation therapy usually is given on an outpatient basis; most patients do not need to stay in the hospital. External radiation therapy is used to treat most types of cancer, including cancer of the bladder, brain, breast, cervix, larynx, lung, prostate, and vagina. In addition, external radiation may be used to relieve pain or ease other problems when cancer spreads to other parts of the body from the primary site.

Intraoperative radiation therapy (IORT) is a form of external radiation that is given during surgery. IORT is used to treat localized cancers that cannot be completely removed or that have a high risk of recurring (coming back) in nearby tissues. After all or most of the cancer is removed, one large, high-energy dose of radiation is aimed directly at the tumor site during surgery (nearby healthy tissue is protected with special shields). The patient stays in the hospital to recover from the surgery. IORT may be used in the treatment of thyroid and colorectal cancers, gynecological cancers, cancer of the small intestine, and cancer of the pancreas. It is also being studied in clinical trials (research studies) to treat some types of brain tumors and pelvic sarcomas in adults.

Prophylactic cranial irradiation (PCI) is external radiation given to the brain when the primary cancer (for example, small cell lung cancer) has a high risk of spreading to the brain.

Internal radiation therapy (also called brachytherapy) uses radiation that is placed very close to or inside the tumor. The radiation source is usually sealed in a small holder called an implant. Implants may be in the form of thin wires, plastic tubes called catheters, ribbons, capsules, or seeds. The implant is put directly into the body. Internal radiation therapy may require a hospital stay. Internal radiation is usually delivered in one of two ways, each of which uses sealed implants. Interstitial radiation therapy is inserted into tissue at or near the tumor site. It is used to treat tumors of the head and neck, prostate, cervix, ovary, breast, and perianal and pelvic regions. Some women treated with external radiation for breast cancer receive a “booster dose” of radiation that may use interstitial radiation or external radiation. Intracavitary or intraluminal radiation therapy is inserted into the body with an applicator. It is commonly used in the treatment of uterine cancer. Researchers are also studying these types of internal radiation therapy for other cancers, including breast, bronchial, cervical, gallbladder, oral, rectal, tracheal, uterine, and vaginal. Systemic radiation therapy uses radioactive materials such as iodine 131 and strontium 89. The materials may be taken by mouth or injected into the body. Systemic radiation therapy is sometimes used to treat cancer of the thyroid and adult non-Hodgkin lymphoma.

### Tumors

Tumors to be treated by the methods and compositions of the present invention may be malignant (e.g., carcinogenic or “cancer”) or benign. Examples of benign tumors for treatment include thyroid adenomas, adrenocortical adenomas, and pituitary adenomas, benign brain tumors (e.g., glioma, astrocytoma, meningioma). By “cancer” is usually meant a group of diseases having the appearance of tumours as symptoms. These tumours are composed of atypical cells, having a capacity for autonomous growth, an imprecise delimitation, an ability to invade neighbouring tissues and vessels and a tendency to disseminate by the production of metastases. Without limitation, examples of cancers which can be treated by the methods and compositions described herein include bladder cancer, melanoma, breast cancer, non-Hodgkin lymphoma, colon and rectal cancer, pancreatic cancer, endometrial cancer, prostate cancer, kidney (renal cell) cancer, skin cancer, (nonmelanoma), leukemia, thyroid cancer, lung cancer, cervical cancer, ovarian cancer, testicular cancer. Primary and metastatic growth of the following tumors can be inhibited by the above-described methods: vulvar epidermoid carcinomas, cervical carcinomas, endometrial adenocarcinomas, ovarian adenocarcinomas and ocular melanomas.

Since metformin can cross the blood brain barrier, it's administration, according to the methods described herein, can be useful in treating or preventing central nervous system tumors, or preventing the spread of cancers to the central nervous system.

The pharmaceutical compositions of this invention may be in the dosage form of solid, semi-solid, or liquid such as, e.g. suspension, aerosols, or the like. Preferably the compositions are administered in unit dosage forms suitable for single administration of precise dosage amounts. The compositions may also include, depending on the formulation desired, pharmaceutically-acceptabl-e, nontoxic carriers or diluents, which are defined as vehicles commonly used to formulate pharmaceutical compositions for animal or human administration. Compositions may be provided as sustained release or timed release formulations. The carrier or diluent may include any sustained release material known in the art, such as glyceryl monostrearate or glyceryl distearate, alone or mixed with a wax. Controlled release preparations can be achieved by the use of polymers to complex or adsorb the metformin and/or chemotherapeutic agent. The controlled delivery can be exercised by selecting appropriate macromolecules (for example polyesters, polyamino acids, polyvinyl pyrrolidone, ethylenevinylacetate, methylcellulose, carboxymethylcellulose, and protamine sulfate) and the concentration of macromolecules as well as the methods of incorporation in order to control release. Microencapsulation may also be used. The timed release formulation can provide a combination of immediate and pulsed release throughout the day. The diluent is selected so as not to affect the biological activity of the combination. Examples of such diluents are distilled water, physiological saline, Ringer's solution, dextrose solution, and Hank's solution. In addition, the pharmaceutical composition of formulation may also include other carriers, adjuvants, emulsifiers such as poloxamers, or nontoxic, nontherapeutic, nonimmunogenic stabilizers and the like. Effective amounts of such diluent or carrier will be those amounts which are effective to obtain a pharmaceutically acceptable formulation in terms of solubility of components, or biological activity, and the like.

Another apsect of the present invention relates to a formulation for treating cancer with the above drug combination. In one embodiment, the formulation includes a controlled-release device where one or several of the drugs are being released in a delayed fashion. Such formulation can be in the form of a tablet (or a pill) which releases different doses of drugs in different time intervals after being taken orally.

Another aspect of the present invention relates to a kit for the treatment of a subject by the methods disclosed herein (e.g., tumor therapy). The kit comprises one or more vials of the metformin and one or more vials of the chemotherapeutic agent(s) (either together or in separate vials), at the doses provided above. The kit may further contain instructions describing their use in combination. The kit may include a formulation of both the metformin together with one or more of the chemotherapeutic agents.

Method for Screening for an Agent that Modulates a Chemotherapeutic Agent or Agents that are Modulated by Metformin

The present invention provides for methods to screen for agents (e.g., metformin derivatives) that modulate chemotherapeutic agents by the methods of the present invention. Tumor, cancer, and/ or cancer stem cells can be used to assay test compounds (e.g., a metformin derivative) for efficacy on killing of the cells. In the methods, a metformin derivative is administered with a known chemotherapeutic agent to the cells, and its ability to kill the cells is determined by measuring an indicating parameter of the cells (e.g., cell viability). The cell viability is compared to an appropriate control which has not received the metformin derivative, and an enhanced killing (e.g., a synergistic effect) indicates that the metformin derivative is an agent that modulates the chemotherapeutic agent.

The present invention also provides for methods to screen for agents which are enhanced in their tumor, cancer, and/or cancer stem cell killing ability, by metformin. In the methods, a test compound is administered with metformin (or an identified metformin derivative) to the cells, and its ability to kill the cells is determined by measuring an indicating parameter of the cells (e.g., cell viability). The cell viability is compared to an appropriate control which has not received the test compound, and an enhanced killing (e.g., a synergistic effect) indicates that the test compound is an agent that is enhanced by metformin.

The test compounds are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method. In some embodiments, agent formulations do not include additional components, such as preservatives, that may have a significant effect on the overall formulation. Thus in one embodiment, formulations consist essentially of a test agent and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

### Test Compounds

The term “ test compound” or “test agent” as used herein and throughout the specification when used in reference to a screening assay, means any organic or inorganic molecule, including modified and unmodified nucleic acids such as antisense nucleic acids, RNAi, such as siRNA or shRNA, peptides, peptidomimetics, receptors, ligands, and antibodies.

The test compound can be any molecule, compound, or other substance which can be administered to a test animal. In some cases, the test agent does not substantially interfere with animal viability. Suitable test compounds may be small molecules, biological polymers, such as polypeptides, polysaccharides, polynucleotides, and the like. The test compounds will typically be administered to the animal at a dosage of from 1 ng/kg to 10 mg/kg, usually from 10 μg/kg to 1 mg/kg. Test compounds can be identified that are therapeutically effective, such as anti-proliferative agents, or as lead compounds for drug development.

In some embodiments, test compound can be from diversity libraries, such as random or combinatorial peptide or non-peptide libraries. Many libraries are known in the art, such as, for example, chemically synthesized libraries, recombinant phage display libraries, and in vivo translation-based libraries.

Examples of chemically synthesized libraries are described in Fodor et al. (Science 251:767-73 (1991)), Houghten et al. (Nature 354:84-86 (1991)), Lam et al. (Nature 354:82-84 (1991)), Medynski (Bio/Technology 12:709-10 (1994)), Gallop et al. (J. Med. Chem. 37:1233-51 (1994)), Ohlmeyer et al. (Proc. Natl. Acad. Sci. USA 90:10922-26 (1993)), Erb et al. (Proc. Natl. Acad. Sci. USA 91:11422-26 (1994)), Houghten et al. (Biotechniques 13:412-21 (1992)), Jayawickreme et al. (Proc. Natl. Acad. Sci. USA 91:1614-18 (1994)), Salmon et al. (Proc. Natl. Acad. Sci. USA 90:11708-12 (1993)), International Patent Publication WO 93/20242, and Brenner and Lerner (Proc. Natl. Acad. Sci. USA 89:5381-83 (1992)).

Examples of phage display libraries are described in Scott and Smith (Science 249:386-90 (1990)), Devlin et al. (Science 249:404-06 (1990)), Christian et al. (J. Mol. Biol. 227:711-18 (1992)), Lenstra (J. Immunol. Meth. 152:149-57 (1992)), Kay et al. (Gene 128:59-65 (1993)), and International Patent Publication WO 94/18318.

In vivo translation-based libraries include, but are not limited to, those described in International Patent Publication WO 91/05058, and Mattheakis et al. (Proc. Natl. Acad. Sci. USA 91:9022-26 (1994)). By way of examples of nonpeptide libraries, a benzodiazepine library (see, e.g., Bunin et al., Proc. Natl. Acad. Sci. USA 91:4708-12 (1994)) can be adapted for use. Peptide libraries (see, e.g., Simon et al., Proc. Natl. Acad. Sci. USA 89:9367-71(1992)) can also be used. Another example of a library that can be used, in which the amide functionalities in peptides have been permethylated to generate a chemically transformed combinatorial library, is described by Ostresh et al. (Proc. Natl. Acad. Sci. USA 91:11138-42 (1994)).

The test agent used in the screening method can be selected from a group of a chemical, small molecule, chemical entity, nucleic acid sequences, an action; nucleic acid analogues or protein or polypeptide or analogue of fragment thereof. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof, can be any protein of interest, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof. The agent may be applied to the media, where it contacts the cell (such as cells of endoderm origin) and induces its effects. Alternatively, the agent may be intracellular within the cell (e.g. cells of endoderm origin) as a result of introduction of the nucleic acid sequence into the cell and its transcription resulting in the production of the nucleic acid and/or protein agent within the cell. An agent also encompasses any action and/or event the cells (e.g. cells of endoderm origin) are subjected to. As a non-limiting examples, an action can comprise any action that triggers a physiological change in the cell, for example but not limited to; heat-shock, ionizing irradiation, cold-shock, electrical impulse, light and/or wavelength exposure, UV exposure, pressure, stretching action, increased and/or decreased oxygen exposure, exposure to reactive oxygen species (ROS), ischemic conditions, fluorescence exposure etc. Environmental stimuli also include intrinsic environmental stimuli defined below. The exposure to agent may be continuous or non-continuous.

In some embodiments, the agent is an agent of interest including known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents also include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Also included as test agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

The agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

Compounds for screening include metformin derivatives and candidate agents (also referred to herein as test agents or test compounds). Candidate agents are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for effect on the cells usually a plurality of tumor/cancer/cancer stem cells, usually in conjunction with like cells lacking the agent. The change in parameters in response to the agent is measured, and the result evaluated by comparison to reference cultures, e.g. in the presence and absence of the agent, obtained with other agents, etc.

Parameters are quantifiable components of cell viability, growth and/or tumorgenesis that can be accurately measured, desirably in a high throughput system. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values. In some embodiments, the assay is a computerized assay or a robotic high-throughput system operated through a computer interface.

Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred test compound is a small molecule, nucleic acid and modified nucleic acids, peptide, peptidomimetic, protein, glycoprotein, carbohydrate, lipid, or glycolipid. Preferably, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection, Bethesda, Md., NCI's Developmental Therapeutics Program, or the like.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

To screen the compounds described above for ability to modulate transcription and/or expression of factors associated with muscle growth, the test compounds should be administered to the test subject. In one embodiment the test subject is a culture of cells comprised of tumor, cancer, or and/or cancer stem cells. The cells may be a primary cell culture or an immortalized cell line from a tumor.

The test compounds can be administered, for example, by diluting the compounds into the medium wherein the cell is maintained, mixing the test compounds with the food or liquid of the animal with muscle, topically administering the compound in a pharmaceutically acceptable carrier on the animal with muscle, using three-dimensional substrates soaked with the test compound such as slow release beads and the like and embedding such substrates into the animal, intramuscularly administering the compound, parenterally administering the compound.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

Preservatives and other additives can also be present. For example, antimicrobial, antioxidant, chelating agents, and inert gases can be added (see, generally, Remington's Pharmaceutical Sciences, 16th Edition, Mack, 1980). As noted above, screening assays are generally carried out in vivo, for example, in cultured cells.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (”consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.
1. A method for treating a tumor in a subject in need thereof comprising administering an enhancing amount of metformin and a reduced amount of one or more chemotherapeutic agents.
2. The method of paragraph 1, wherein the enhancing amount of metformin is 250 mg/day.
3. A composition comprising an enhancing amount of metformin, and a reduced amount of one or more chemotherapeutic agents and a pharmaceutically acceptable carrier.
4. The composition of paragraph 3, wherein the enhancing amount of metformin is about 25 mg.
5. The composition of paragraph 3, wherein the enhancing amount of metformin is about 75 mg.
6. The composition of paragraph 3, wherein the enhancing amount of metformin is about 250 mg.
7. A kit comprising a vial of metformin, a vial of one or more chemotherapeutic agents, instructions for the use of the metformin and the chemotherapeutic agent(s) together.
8. A method for preventing cancer or delaying the recurrence of cancer in a subject comprising administering an effective amount of metformin to the subject.
9. The method of paragraph 8, wherein the amount of metformin is about 75 mg/day.

The invention is further illustrated by the following examples, which should not be construed as further limiting.

### EXAMPLES

### Example 1

Here, it is shown that metformin selectively kills cancer stem cells in four genetically different types of breast cancer. The combination of metformin and doxorubicin, a well-defined chemotherapeutic drug, kills both cancer stem cells and non-stem cancer cells in culture, and reduces tumor mass and prolongs remission much more effectively than either drug alone in a xenograft mouse model. These observations constitute independent support for the cancer stem cell hypothesis, and they provide a rationale for why the combination of metformin and chemotherapeutic drugs might improve treatment of patients with breast (and possibly other) cancers.

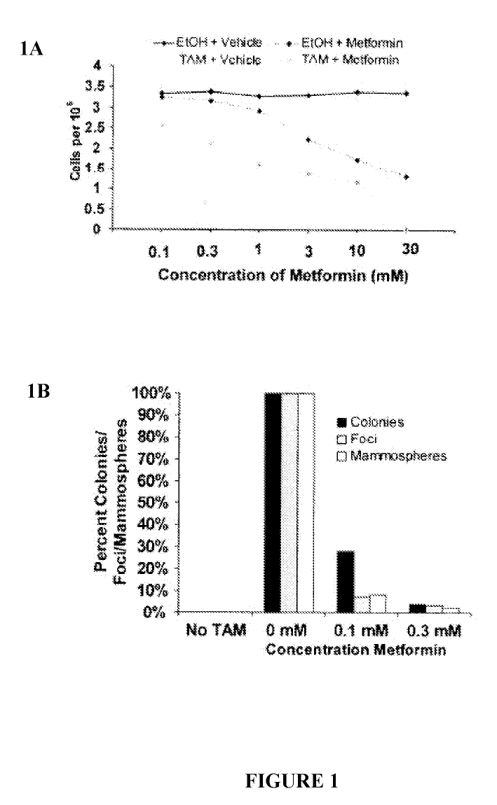

To examine the anti-cancer properties of metformin, we first utilized an inducible transformation model consisting of non-transformed human mammary epithelial cells (MCF-10A) containing ER-Src, a fusion of the v-Src oncoprotein with the ligand-binding domain of estrogen receptor. When these cells are treated with tamoxifen, they become transformed within 24-36 hours. The transformed cell population contains 10% cancer stem cells, as defined by expression of the CD44 marker and the ability to form mammospheres, multicellular “micro-tumors” that are generated in non-adherent and non-differentiating conditions (18). In addition, we analyzed three other mammary adenocarcinoma cell lines derived from genetically and phenotypically different tumors that are treated with different drugs: ER-positive MCF7 (13); HER-positive SKBR3 (14); triple-negative MDA-MB-468 (15). These cell lines also contain a minority population of cancer stem cells capable of mammosphere formation. In all experiments, metformin was used at a concentration that does not affect the growth of non-transformed cells (0.1 or 0.3 mM; FIG. 1A). Previous experiments on cancer cell lines (7-9) used much higher concentrations of metformin (typically 10-30 mM), conditions that are also toxic for non-transformed cells.

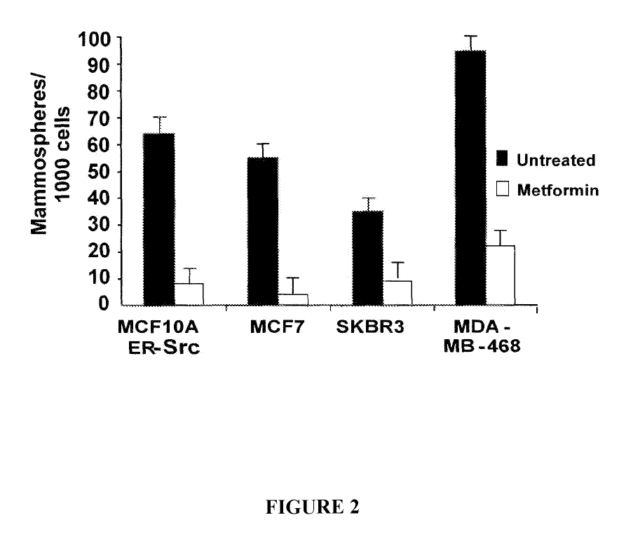

In the inducible MCF-10A model, metformin strongly inhibited morphological transformation, as seen in phase-contrast images of cells grown in the presence or absence of 0.1 mM metformin and/or TAM for 36 hours (data not shown), invasive growth in wound-healing assays, as seen in wound-healing/invasion response assay of cells grown in the presence or absence of 0.1 mM metformin and/or TAM (data not shown), focus formation, formation of colonies in soft agar, and generation of mammospheres (FIG. 1B). Furthermore, metformin treatment of mammospheres derived from all four breast cancer cell lines caused a dramatic reduction in the number of mammospheres within 48 hours (FIG. 2) as a consequence of cell death. As mammospheres are composed primarily of cancer stem cells (18), this latter observation suggests that metformin may kill cancer stem cells.

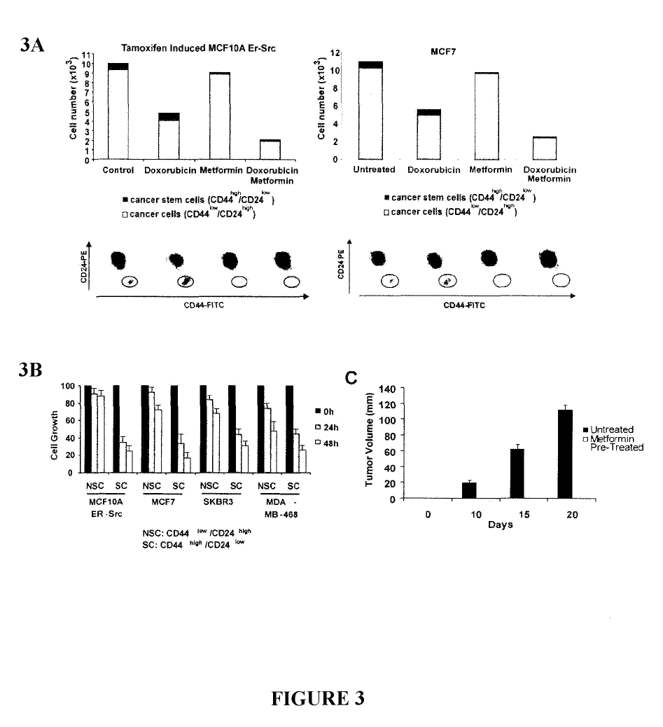

Strikingly, metformin preferentially killed cancer stem cells (CD44high/CD24low) within a population of transformed MCF-10A or MCF-7 cells (FIG. 3A). Similarly, when all four cancer cell lines were sorted, cancer stem cells were quite susceptible to metformin, whereas the standard cancer cell population remains essentially unaffected (FIG. 3B). Furthermore, treatment of MCF-10A cancer stem cells with metformin for just 1 hour blocked the ability of these cells to form tumors in nude mice, even though the drug was not present for the month after injection (FIG. 3C). The ability of metformin to selectively kill cancer stem cells was in marked contrast to doxorubicin, a chemotherapeutic agent that kills cancer cells, but not cancer stem cells. As expected from their distinct properties, metformin worked together with doxorubicin to reduce both non-stem cancer cells and cancer stem cells in the mixed transformed population (FIG. 3A).

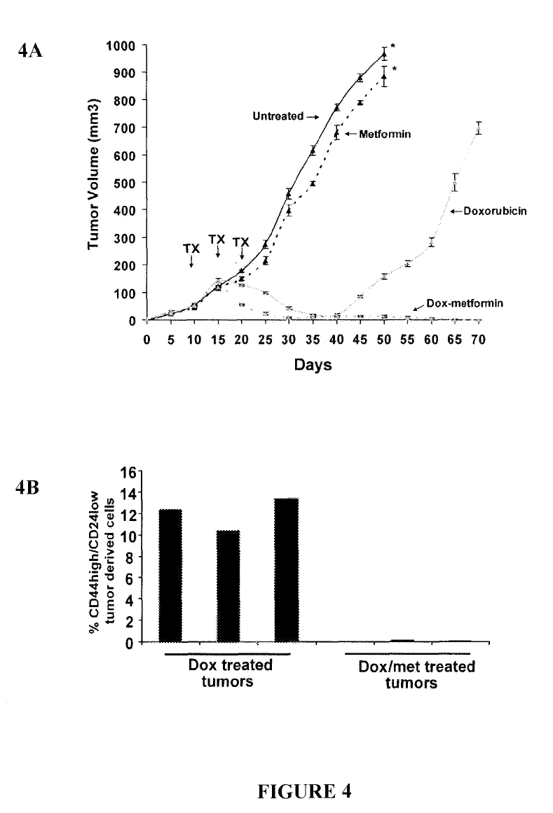

In accord with the above results in cell lines, the synergy between metformin and doxorubicin was observed upon treatment of tumors that arise 10 days after injection of MCF-10A-ER-Src cells into nude mice. After 15 days of treatment (3 cycles every 5 days), this drug combination virtually eliminated tumors, whereas doxorubicin alone caused only a 2-fold decrease in tumor volume and metformin alone has little effect (FIG. 4A). Doxorubin-treated mice showed a further reduction in tumor volume after an additional 10 days (day 35). The minimal effect of metformin alone was in contrast to more significant effects seen in an independent report (8), but there were many differences in experimental protocol between these studies.

To determine the basis for why the combination of metformin and doxorubicin is more effective than doxorubicin alone, we examined the population of cells recovered from tumors after 3 cycles of treatment (day 25). In accord with our results in cell lines, cancer stem cells were virtually absent from mice treated with the drug combination, whereas they were easily detected in tumors from mice treated with doxorubicin alone (FIG. 4B). Thus, the therapeutic advantage of metformin in the context of conventional chemotherapy is linked to its ability to kill cancer stem cells.

The cancer stem cell hypothesis for the progression of human disease is based on the differential tumor-forming properties and responses to well-defined chemotherapy of cancer stem cells and non-stem cancer cells. A prediction of this model, heretofore untested, is that drugs that selectively inhibit cancer stem cells should function synergistically with chemotherapeutic drugs to delay relapse. Strikingly, mice treated with the combination of metformin and doxorubicin remained in remission for at least 60 days after treatment was ended (FIG. 4A). In contrast, tumor growth resumed 20 days after mice were treated with doxorubicin alone, and the rate of tumor growth after relapse was comparable to that observed in the initial disease (i.e. in the absence of treatment). Thus, combinatorial therapy had a dramatic effect on prolonging remission, and indeed may even represent a cure of these xenograft-generated tumors. In addition to their potential medical significance, these observations provide independent and further support for the cancer stem cell hypothesis.

To our knowledge, the ability of metformin to selectively kill cancer stem cells and to function synergistically with doxorubicin to block both cancer stem cells and non-stem transformed cells is unique. In the case of breast cancer, herceptin and tamoxifen are useful drugs for cancer types that, respectively, express the HER2 and estrogen receptors, but some forms of breast cancer lack these receptors resist these treatments. For all of these types of breast cancer, metformin selectively inhibits cancer stem cell growth, and hence is likely to function synergistically with chemotherapeutic drugs. In addition, as metformin inhibited transformation of MCF10A-ER-Src cells, suggesting that it has the ability to prevent the development of cancer, as opposed to treating cancer that has already occurred. Indeed, the ability of metformin to inhibit cellular transformation might underlie the epidemiological observation that diabetics treated with metformin have a lower incidence of cancer (5, 6). As a cancer preventative, metformin is preferably administered on a long-term basis, and in this regard, the concentration of metformin needed for the anti-cancer effects observed here is considerably below that used for the treatment of diabetes. Lastly, the selectivity of metformin and doxorubicin for distinct types of cells in the tumor can explain the striking combinatorial effects on reducing tumor mass and prolonging remission in nude mice, and it provides the rationale for combining metformin with chemotherapy as a new treatment for breast or other cancers.

### Methods of the Invention

### Cell Lines

MCF10A cells are mammary epithelial cells derived from fibrocystic breast tissue that was obtained from a mastectomy of a 36-years old woman with no family history of breast cancer and no evidence of disease (12). Genetic analysis did not reveal any amplification of HER2/neu oncogene or mutations in H-Ras oncogenes, and these cells do not express estrogen receptor. The experiments here use a derivative of MCF10A containing an integrated fusion of the v-Src oncoprotein with the ligand binding domain of estrogen receptor. MCF7 cells are mammary adenocarcinoma cells that express very high levels the estrogen receptor, are negative for HER2/neu, and do not have strong anchorage-independent properties (13). SKBR3 cells are mammary adenocarcinoma cells that overexpress the HER2/neu receptor, have anchorage-independent properties, and form tumors in xenografts (14). MDA-MB-468 cells are derived from a triple negative breast carcinoma that shows many of the recurrent basal-like molecular abnormalities including ER-PR-HER2-negative status, p53 deficiency, EGFR overexpression, PTEN loss and constitutive activation of the MEK/ERK pathway (15). MDA-MB-468 cells are very aggressive and form large tumors in xenograft experiments that resist treatment with tamoxifen or herceptin.

### Cell Culture

MCF-7, SKBR3, and MDA-MB-486 cells were grown in DMEM media (Invitrogen), 10% fetal bovine serum (Atlanta Biologicals), and penicillin/streptomycin (Invitrogen) at 37° C. with 5% CO₂. MCF10A ER-Src cells were cultured as described previously (16) and induced to transform with 1 μM 4OH-tamoxifen (TAM) dissolved (Sigma) in EtOH. Morphological changes, phenotypic transformation and foci formation occurred 24-36 h after TAM addition, and were monitored by phase-contrast microscopy. Metformin (Sigma) dissolved in water was typically added to 0.1 mM unless otherwise indicated.

### Wound Healing Motility Assay

Cells were seeded onto six-well dishes at 1×10⁵/well. A single scratch wound was created using a p10 micropipette tip in to confluent cells. Cells were washed three times with PBS to remove cell debris, supplemented with assay medium, and monitored. Images were captured by phase-contrast microscopy at 0 and 12 h post wounding.

### Colony Formation Assay

Triplicate samples of 5×10⁴ cells from MCF10A ER-Src were mixed 4:1 (v/v) with 2.0% agarose in MCF-10A growth medium for a final concentration of 0.4% agarose. The cell mixture was plated on top of a solidified layer of 0.5% agarose in growth medium. Cells were fed every 6 to 7 days with growth medium containing 0.4% agarose. The number of colonies was counted after 15 days.

### Mammosphere Culture

Mammospheres were cultured in suspension (1000 cells/ml) in serum-free DMEM/F12 media, supplemented with B27 (1:50, Invitrogen), 0.4% BSA, 20 ng/ml EGF (Preprotech) and 4 μg/ml insulin (Sigma) as described previously (17). Mammosphere formation was tested by placing transformed cell populations in the presence of absence of metformin under these conditions, whereas mammosphere growth was examined by adding metformin to 6-day old mammospheres and counting the number of mammospheres 2 and 4 days after treatment.

### Isolation and Analysis of Cancer Stem Cells

Flow cytometric cell sorting of transformed cell populations was performed on single cell suspensions. Cells were stained with CD44 antibody (FITC-conjugated) (555478, BD Biosciences) and with CD24 antibody (PE-conjugated) (555428, BD Biosciences). Cancer stem cells (CD44high/CD24low) and no-stem transformed cells (CD44low/CD24high) from MCF10A ER-Src (TAM-treated) and MCF7, SKBR3 and MDA-MD-486 cells were treated with 0.1 mM metformin and cell growth was assessed in different time points (12, 24, 48 h). The experiments were performed in triplicate, and the data represent mean±SD.

### Tumor Growth and Relapse in Xenografts

5×106 MCF10A ER-Src cells were injected into the right flank of 16 female nu/nu mice (Charles River Laboratories), all of which developed tumors in 10 days with size ˜50 mm3. The mice were randomly distributed into 4 groups that were untreated, or treated by intraperitoneal injections every 5 days (3 cycles) with 4 mg/kg doxorubicin, 100 μg/ml metformin, or the combination. Tumor volume (mean values and 95% confidence intervals) was measured at various times after the initial injection. All the mouse experiments were performed in accordance with Institutional Animal Care and Use Committee procedures and guidelines.

The references cited herein are incorporated by reference.

### REFERENCES

1. Ailles L E, Weissman I L. Cancer stem cells in solid tumors. Curr Opin Biotechnol 2007; 18:460-6.
2. Polyak K, Weinberg R A. Transitions between epithelial and mesenchymal states: acquisition of malignant and stem cells traits. Nat Rev Cancer 2009; 9:265-73.
3. Larsson S C, Mantzoros C S, Wolk A. Diabetes mellitus and risk of breast cancer: a meta-analysis. International journal of cancer 2007; 121:856-62.
4. Hsu I R, Kim S P, Kabir M, Bergman R N. Metabolic syndrome, hyperinsulinemia, and cancer. Am J Clin Nutr 2007; 86:867-71.
5. Evans J M, Donnelly L A, Emslie-Smith A M, Alessi D R, Morris A D. Metformin and reduced risk of cancer in diabetic patients. BMJ (Clinical research ed 2005; 330:1304-5.
6. Jiralerspong S, Palla S L, Giordano S H, et al. Metformin and pathologic complete responses to neoadjuvant chemotherapy in diabetic patients with breast cancer. J Clin Oncol 2009; 27:3297-302.
7. Alimova I N, Liu B, Fan Z, et al. Metformin inhibits breast cancer cell growth, colony formation and induces cell cycle arrest in vivo. Cell Cycle 2009; 8:909-15.
8. Liu B, Fan Z, Edgerton S M, et al. Metformin induces unique biological and molecular responses in triple negative breast cancer cells. Cell Cycle 2009; 8:2031-40.
9. Zakikhani M, Dowling R, Fantus I G, Sonenberg N, Pollak M. Metformin is an AMP kinase-dependent growth inhibitor for breast cancer cells. Cancer research 2006; 66:10269-73.
10. Cazzaniga M, Bonanni B, Guerrieri-Gonzaga A, Decensi A. Is it time to test metformin in breast cancer clinical trials? Cancer Epidemiol Biomarkers Prey 2009; 18:701-5.
11. Goodwin P J, Ligibel J A, Stambolic V. Metformin in breast cancer: Time for action. J Clin Oncol 2009; 27:3271-3.
12. Soule H D, Maloney T M, Wolman S R, et al. Isolation and characterization of a spontaneously immortallized human breast epithelial cell line, MCF10. Cancer research 1990; 50:6075-86.
13. Brooks S C, Locke E R, Soule H D. Estrogen receptor in a human cell line (MCF-7) from breast carcinoma. J Biol Chem 1973; 248:6251-3.
14. Bergman I, Barmada M A, Griffin J A, Slamon D J. Treatment of meningeal breast cancer xenografts in the rat using an anti-p185/HER2 antibody. Clin Cancer Res 2001; 7:2050-6.
15. Oliveras-Ferraros C, Vazquez-Martin A, Lopez-Bonet E, et al. Growth and molecular interactions of the anti-EGFR antibody cetuximab and the DNA crosslinking agent cisplatin in gefitinib-resistant MDA-MB-468 cells: new prospects in the treatment of triple-negative/basal-like breast cancer. Int J Oncol 2008; 33:1165-76.
16. Debnath J, Muthuswamy S K, Brugge J S. Morphogenesis and oncogenesis of MCF-10A mammary epithelial acini grown in three-dimensional basement membrane cultures. Methods 2003; 30(3):256-68.
17. Dontu G, Abdallah W M, Foley J M, et al. In vivo propagation and transcriptional profiling of human mammary stem/progenitor cells. Genes Dev 2003; 17:1253-70.
18. Grimshaw M J, Cooper L, Papazisis K, et al. Mammosphere culture of metastatic breast cancer cells enriches for tumorigenic breast cancer cells. Breast Cancer Res 2008;10:R52.

What is claimed:

1. A method for treating a tumor in a subject in need thereof comprising administering an enhancing amount of metformin and a reduced amount of one or more chemotherapeutic agents.

2. The method of claim 1, wherein the enhancing amount of metformin is 250 mg/day.

3. A composition comprising an enhancing amount of metformin, and a reduced amount of one or more chemotherapeutic agents and a pharmaceutically acceptable carrier.

4. The composition of claim 3, wherein the enhancing amount of metformin is about 25 mg.

5. The composition of claim 3, wherein the enhancing amount of metformin is about 75 mg.

6. The composition of claim 3, wherein the enhancing amount of metformin is about 250 mg.

7. A kit comprising a vial of metformin, a vial of one or more chemotherapeutic agents, instructions for the use of the metformin and the chemotherapeutic agent(s) together.

8. A method for preventing cancer or delaying the recurrence of cancer in a subject comprising administering an effective amount of metformin to the subject.

9. The method of claim 8, wherein the amount of metformin is about 75 mg/day.
